(19) United States
(12) Patent Application Publication
(10) Pub. No.: US 2017/0148359 A1
(43) Pub. Date: May 25, 2017

(54) VISUALLY CUED LABELING

(71) Applicant: Stryker European Holdings I, LLC, Kalamazoo, MI (US)

(72) Inventors: Orrin R. Levine, Wyckoff, NJ (US); Benjamin Melinger, New York, NY (US)

(21) Appl. No.: 14/947,292

(22) Filed: Nov. 20, 2015

(51) Int. Cl. G09F 3/02; A61B 17/86; G09F 3/00

(52) U.S. Cl. CPC G09F 3/02; G09F 3/0288; A61B 17/865; G09F 2003/0272; G09F 2003/0208; G09F 2003/023

(57) ABSTRACT

A medical device package label system includes a plurality of indicators which provide information regarding the device in the package. The indicators include images of the device. The size, position, color, or opacity of the indicators provides information regarding features of the device in the package to allow a user to quickly identify the contents of the package. The system also combines letters and numbers with colors and shapes to provide information to the user.

### BACKGROUND OF THE INVENTION

The present invention relates to a labeling system and more particularly, to a visually cued labeling system for use with medical products.

Labels on medical product packages provide critical information to the clinician or user. Some of the information is required by various government agencies, most notably the Federal Food and Drug Administration (FDA) who requires such packaging to include information to trace products back to their manufacturers in case of malfunction, expiration dates, and other information related to the medical product. Other information may also be included to appeal to consumers, tout the benefits of a product, or for aesthetic reasons. Including all of this information in printed form on a label can make it difficult to easily distinguish between medical devices in similar containers. This problem is further complicated when a line of products have similar features which only vary slightly. Therefore, a need exists for an improved labeling system which is readily visible and legible without increasing the overall size of the device package.

### BRIEF SUMMARY OF THE INVENTION

One aspect of the present invention is a labeling system which desirably includes a first indicator and a second indicator. The first indicator may be representative of a medical device in a container and the second indicator may signify a first feature of the medical device. The first indicator can include an image of the medical device. The size and/or the color of the second indicator can indicate one or more dimensions of the medical device. The second indicator may be formed by one or more colored markers with at least one of the colored markers having a different opacity than another of the colored markers. The label can have an adhesive backing configured to be secured to the container. The label may include a third indicator, and possibly a fourth indicator, which each signify at least one additional feature of the medical device. The label may also include a fifth indicator signifying a use-by date.

Another aspect of the present disclosure describes a labeling system which preferably includes a first indicator representative of a medical device and a second indicator representative of a first feature of the medical device. The label can have an edge and the distance separating the second indicator from the edge can signify a dimension of the first feature of the medical device. The first indicator can be an image of the medical device which reflects the quantity of devices in the container. The second indicator may include a colored marker, wherein the color indicates a dimension of the device. The label may also include a third indicator and a fourth indicator which each signify at least one feature of the device.

### BRIEF DESCRIPTION OF THE DRAWINGS

FIG. 1 illustrates a label for a product in accordance with one embodiment of the present disclosure.

FIG. 2 illustrates the label of FIG. 1 having at least one different indicator for a different product.

FIG. 3 illustrates the label of FIG. 1 having at least one different indicator for a different product.

FIG. 4 illustrates the label of FIG. 1 having at least one different indicator for a different product.

FIG. 5 illustrates the label of FIG. 1 having at least one different indicator for a different product.

FIG. 6 illustrates the label of FIG. 1 having at least one different indicator for a different product.

FIG. 7 illustrates the label of FIG. 1 having at least one different indicator for a different product.

FIG. 8 illustrates a plurality of containers having the labels of FIGS. 1-3 and 5-7.

FIG. 9 illustrates a label for a product in accordance with one embodiment of the present invention.

FIG. 10 illustrates the label of FIG. 9 with the marker in a different position for a different product.

FIG. 11 illustrates the label of FIG. 9 with the marker in a different position for a different product.

FIGS. 12A-12C illustrate the label of FIG. 1 having at least one different indicator for a different product.

FIGS. 13A-13C illustrate the label of FIG. 1 having at least one different indicator for a different product.

FIGS. 14A-14B illustrate the label of FIG. 1 having at least one different indicator for a different product.

FIGS. 15A-15B illustrate the label of FIG. 1 having at least one different indicator for a different product.

### DETAILED DESCRIPTION

The labeling system described herein includes indicators which provide a way for a user to visually identify a medical device in a package as well as some or all of the properties of the device relatively quickly. The system combines visual indicators such as shapes and colors with letters and numbers to provide a system which is readily visible and legible for users to distinguish the features of the product in the package from other similar products.

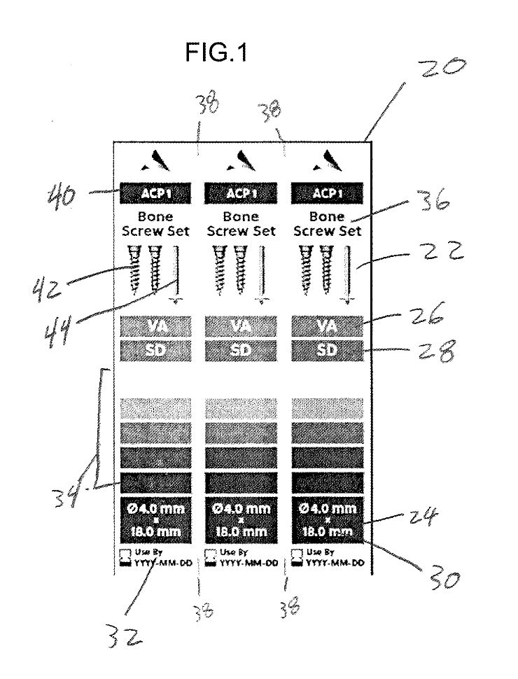

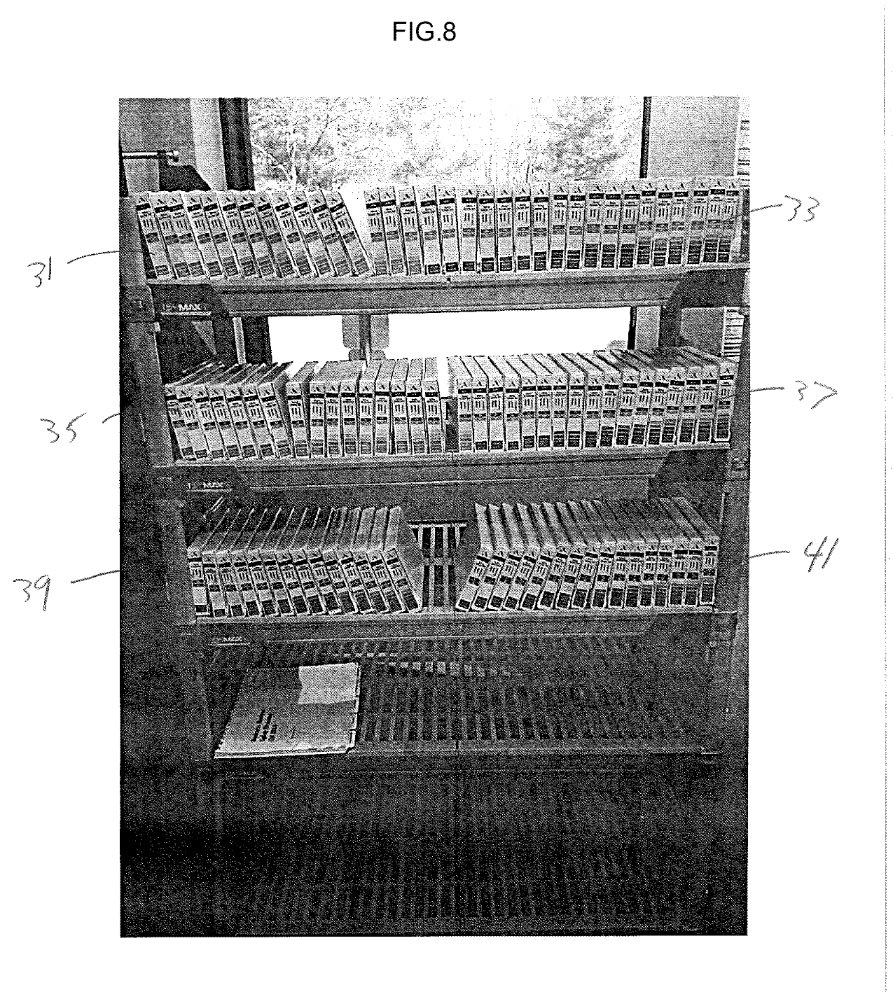

FIG. 1 illustrates a label 20 in accordance with one embodiment of the present invention which is designed to be applied to a package containing a medical device in the form of a bone screw set. The label of FIG. 1 has an adhesive backing such that the label can be applied to a package after printing. Of course, the label could also be directly printed on the package itself. The label 20 includes a first indicator or image 22 representative of a medical device contained in a package. For instance, the image 22 on the label of FIG. 1 shows a bone screw set including two bone screws 42 and a locking screw 44, which references the type and quantity of the medical device in the package allowing a user to relatively quickly glean the contents of the package without the need to fully analyze the label. The image 22 is also scaled to reflect the relative size of the bone screw set. Thus, a user can select a desired size bone screw set from a group of packages each containing bone screw sets as shown in FIG. 8 by visually scanning the image 22. However, the image 22 need not be the only indicator which reflects the size of the medical device in the package. Of course, the labeling system can be used for any type of device in the package (e.g. interbody devices, hip prosthesis, bone plates, expandable implants).

The label of FIG. 1 also includes a second indicator or marker 24. The marker 24 is colored to indicate the diameter of the bone screw 42 in the package. Different colored markers can be used to indicate different diameters. For example, a blue marker could indicate a smaller diameter while a green marker could indicate a larger diameter.

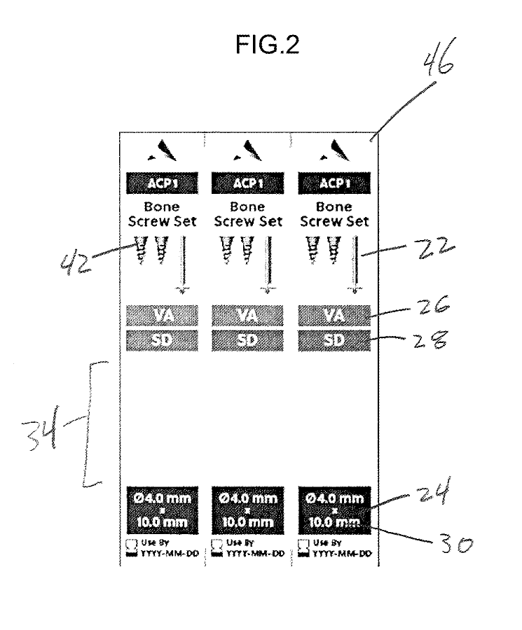

The label 20 has additional markers 34 which illustrate the length of the bone screw 42. The additional markers 34 are of varying opacity to further assist a user in identifying the length of the bone screw. The quantity of additional markers 34 or the total height of the additional markers can indicate the length of the bone screw 42. For example, the additional markers 34 on the label of FIG. 1 indicate an 18 mm bone screw while the absence of additional markers on the label of FIG. 2 indicates a 10 mm bone screw. It is believed that varying the opacity of the additional markers assists the user in discerning, from a distance, the number of additional markers present. For example, a user familiar with the label system can recognize that a marker of certain opacity corresponds to a known number of additional markers. The additional markers could be used to indicate any desired property of a medical device (e.g. height, diameter, porosity, strength, material composition, foot print of an interbody device). The label of FIG. 1 further includes an alpha-numeric indicator 30 which displays the diameter and length of the bone screw 42 of the bone screw set.

The label 20 of FIG. 1 has third and fourth indicators 26, 28 to provide further classification information regarding the implant. As shown, the third indicator 26 is a colored marker with the letters “VA” thereon to indicate that the screw can be inserted into a bone plate at varying angles, and the fourth indicator 28 is also a colored marker and includes the letters “SD” to indicate that the bone screw is self-drilling. The colors of the second, third, and fourth indicators 24, 26, 28 are contrasting colors to allow a user to distinguish between the colors with relative ease. Of course any combination of letters or numbers can be utilized as long as the user is made aware of the meaning of such indicia.

The label 20 includes letters and numbers indicating the implant group title 36, the product title 40, and an expiration date 32. In other words, the label is not restricted to conveying information by the indicators alone, but instead, can also spell out some or all of the information signified by the indicators. The label 20 includes three faces separated by fold lines 38. The three faces allow the label to extend around the sides of a package when the label is folded along the fold lines 38 and adhered to the package such that the user can read the label from 3 sides. The three faces in the embodiment of FIG. 1 include the same information and indicators. However, the faces are not required to be identical to one another and each face could display different information if desired.

FIG. 2 illustrates a label 46 which includes some of the features of the label 20 in FIG. 1. However, the bone screw meant to be identified by the label shown in FIG. 2 has a different length than that of FIG. 1. The image 22 of the bone screw reflects the smaller size of the bone screw when compared to the image of FIG. 1. By comparing the images of FIGS. 1 and 2, a user is able to quickly determine which label contains the larger bone screw. The label 46 does not show any additional markers 34. This absence of additional markers also reflects the smaller length bone screw as compared to that of FIG. 1 and further signifies that this is the smallest bone screw in this line of products. The alpha-numeric indicator 30 displays information to provide further confirmation about the dimensions of the bone screw in addition to the indicators.

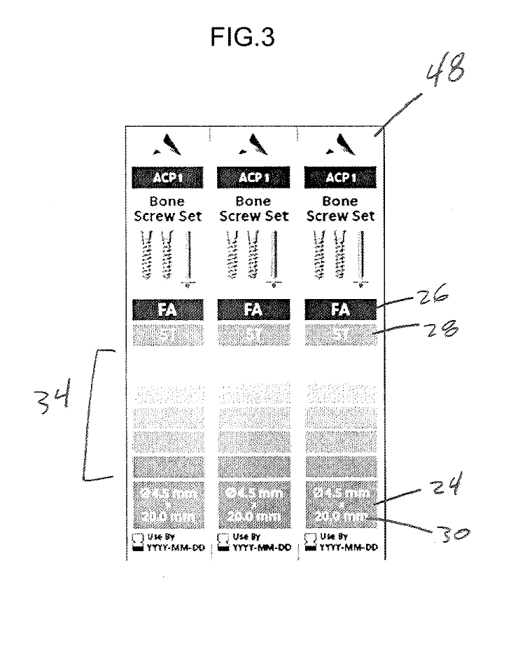

FIG. 3 illustrates a label 48 for a bone screw set with a bone screw having a different diameter than the bone screw of FIG. 1. The different diameter is indicated by the different color of the colored marker 24 as compared to FIG. 1. The number of additional markers 34 indicates a bone screw having a different length than that of FIG. 1. The additional markers 34 occupy all of the space between the fourth indicator 28 and colored marker 24 indicating that the bone screw is the largest offered in the product line. The third indicator 26 is a different color than that of FIG. 1 and includes the letters “FA” to indicate a fixed angle screw. The fourth indicator 28 is a different color than that of FIG. 1 and includes the letters “ST” to indicate a self-tapping screw. The combination of letters and colors on the third and fourth indicators 26, 28 provide confirmation regarding the properties of the screw.

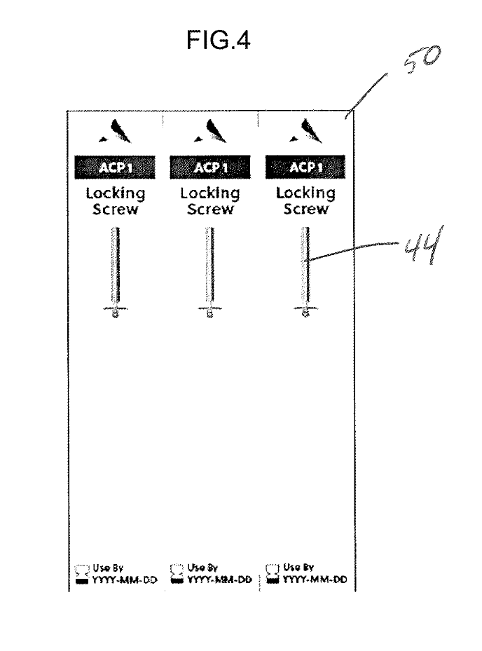

FIG. 4 illustrates a label 50 for a locking screw 44. The locking screw 44 is used with the bone screw set of FIG. 1 and has standard dimensions. Thus, the label 50 does not have any additional markings showing dimensions. It is believed that the absence of markings helps a user identify the locking screw package. Of course, if more than one locking screw is offered the indicators 22, 24, 26, 28 could be used to display the features of the locking screw. Similarly, the label system can be used with any medical device and the indicators can be used to convey any desired feature differentiation of the devices.

For example, FIG. 5 illustrates a label 52 that is designed for use with a cervical plate package, as is easily identified by the inclusion of an image 22 of a cervical plate 54. The cervical plate 54 includes a first level 56 and a second level 58. The color of the third indicator 26 signifies the number of levels of the cervical plate 54. One possible coloring scheme for the third indicator of FIG. 5 is a plate with one level is blue, two levels is purple, three levels is pink, and four levels is yellow. The number of levels is also written on the third indicator 26. The additional markers 34 illustrate the comparative length of the plate 54 within a level. For example, FIG. 5 illustrates additional markers for a plate which is the largest two level plate. Its overall length is shown on the second indicator 24.

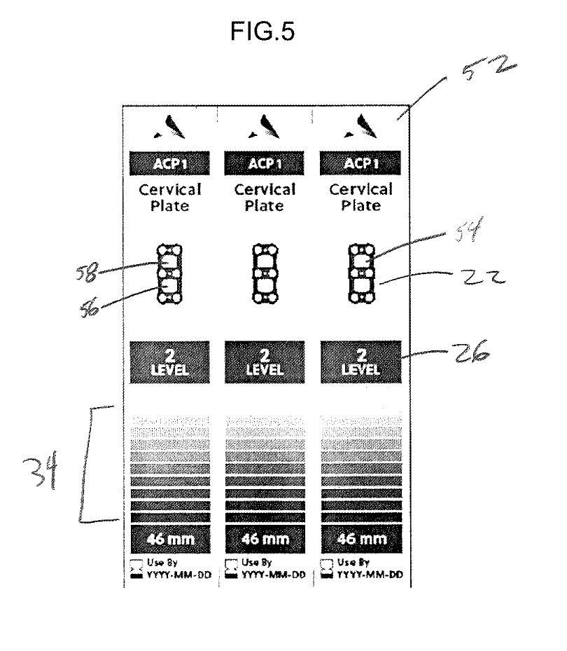

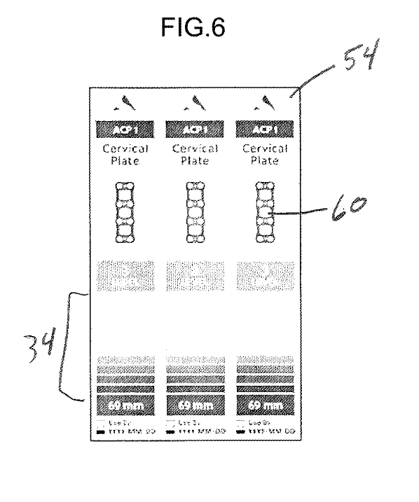

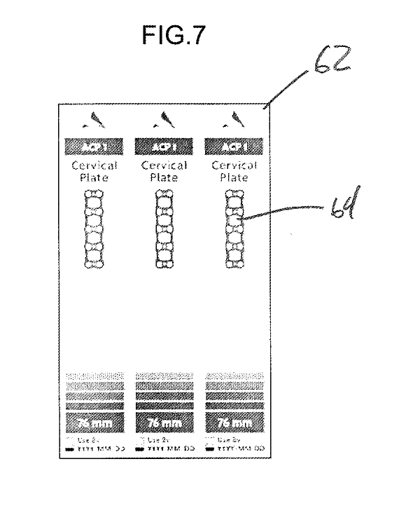

The label 54 of FIG. 6 is for a plate 58 having 3 levels. Although the overall length of the plate 58 in FIG. 6 is greater than that of FIG. 5, the number of additional markers is less than that of FIG. 5 because the number of additional markers indicates the relative length of the plate within its level class. The label 62 of FIG. 7 shows a cervical plate 64 having 4 levels. The labels of FIGS. 5-7 accommodate a greater number of additional markers 34 than the labels of FIGS. 1-4 because there are more gradations in the physical property represented by the additional markers of FIGS. 5-7 than that of FIGS. 1-2. In other words, any number of additional markers 34 can be included in the label system to reflect any number of variations of a property of a medical device. The width of the cervical plate can be indicated by the color of the marker 24.

FIG. 8 illustrates multiple labels affixed to containers which allow a user to visually observe the physical properties of the medical device within the container without the need to read all (or in some cases, any) of the alpha-numeric indicators on the label. The devices are grouped together according to their features indicated by the second, third, and fourth indicators previously described. For example, the first group 31 includes fixed angle, self-tapping, 4.5 mm screws as indicated by the third and fourth indicators 26, 28 on the label 48 as discussed in regards to FIG. 3. The remaining groups 33, 35, 37, 39, 41 are grouped together to have similar features where only the length varies. The number of additional markers 34 increases from zero to the maximum amount from a left to right direction. Thus, a user can select the desired length bone screw by first examining the indicators 22, 24, 26, 28 to select the desired features and then scanning the group 31 to pick the size. Grouping like-featured products in this manner assists the clinician to identify when a product is out of stock and needs to be replaced. Of course, the product groupings could be done in any number of ways. For example, screws having the same length but different features could be grouped together and the clinician could first identify length and then select the features, if desired. Other combinations are also possible. The label system also assists a clinician to refile the unused packages in their proper place based on the color of the labels, height or number of markings, etc. The labels provide easy identification of when a package is misplaced on a storage shelf.

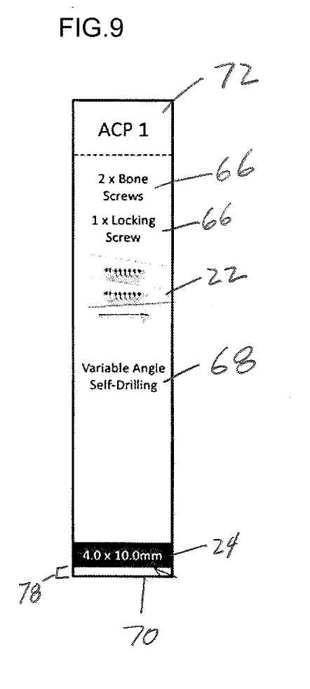

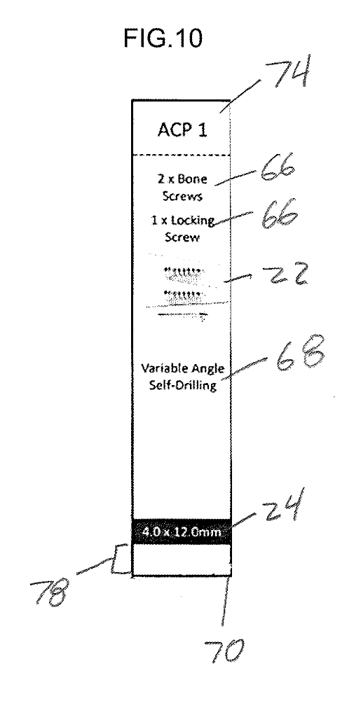

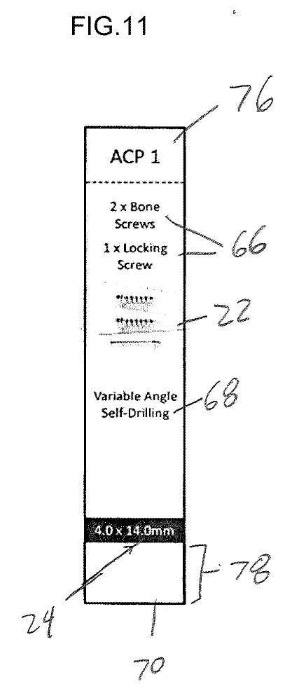

FIGS. 9-11 illustrate another embodiment of a labeling system. Some elements of FIGS. 9-11 are similar to that of FIGS. 1-7 and like reference numerals are used. For example, the label 72 of FIG. 9 includes an image 22 of the device in the package. The label 72 includes a quantity list 66 which indicates the contents of the package, and a colored marker 24 which follows the color scheme previously described to indicate the diameter of the bone screw. However, the label 72 does not include the additional markers of FIGS. 1-7. Instead the distance 78 of the colored marker 24 from an edge 70 of the label 72 indicates the length of the bone screw. For example, as illustrated in the labels 72, 74, 76 of FIGS. 9-11, respectively, the distance of the colored marker 24 from the edge 70 increases as the length of the bone screw increases. Thus, a user can quickly visually ascertain the relative length of the bone screw without reading the alpha-numeric indicator.

The label 72 includes a feature list 68 that describes the bone screw as variable angle and self-drilling. Of course, the third and fourth indicators 26, 28 could be incorporated into the label 72. Similarly, any combination of the features of the label of FIGS. 1-7 could be adopted into the label of FIGS. 9-11.

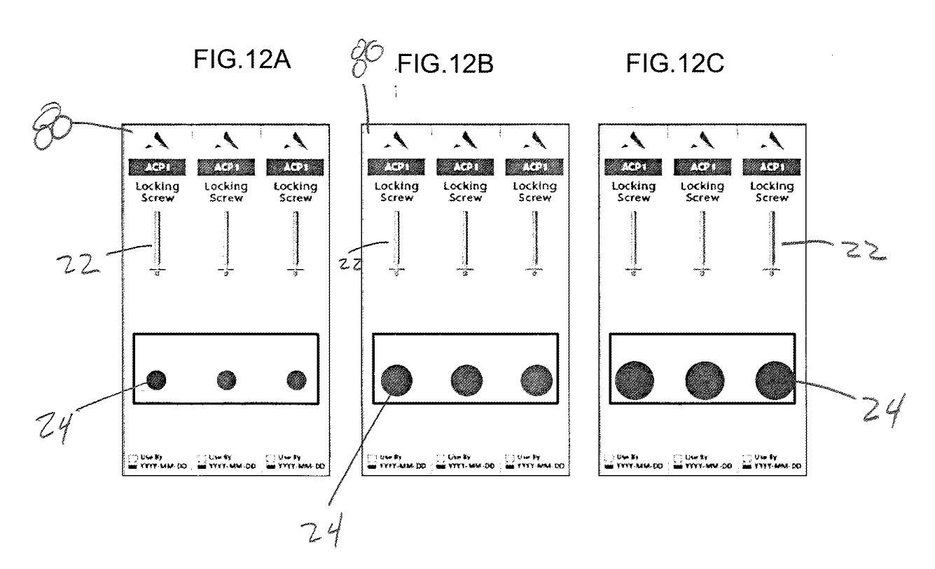

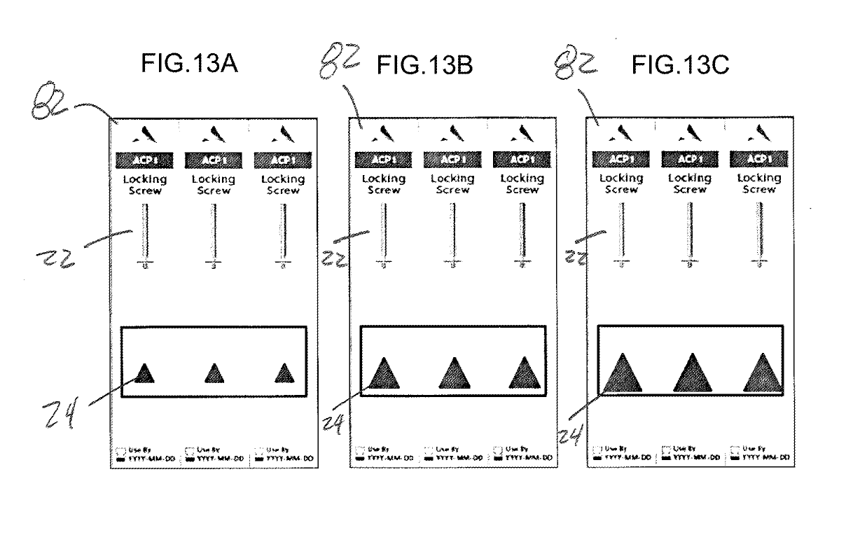

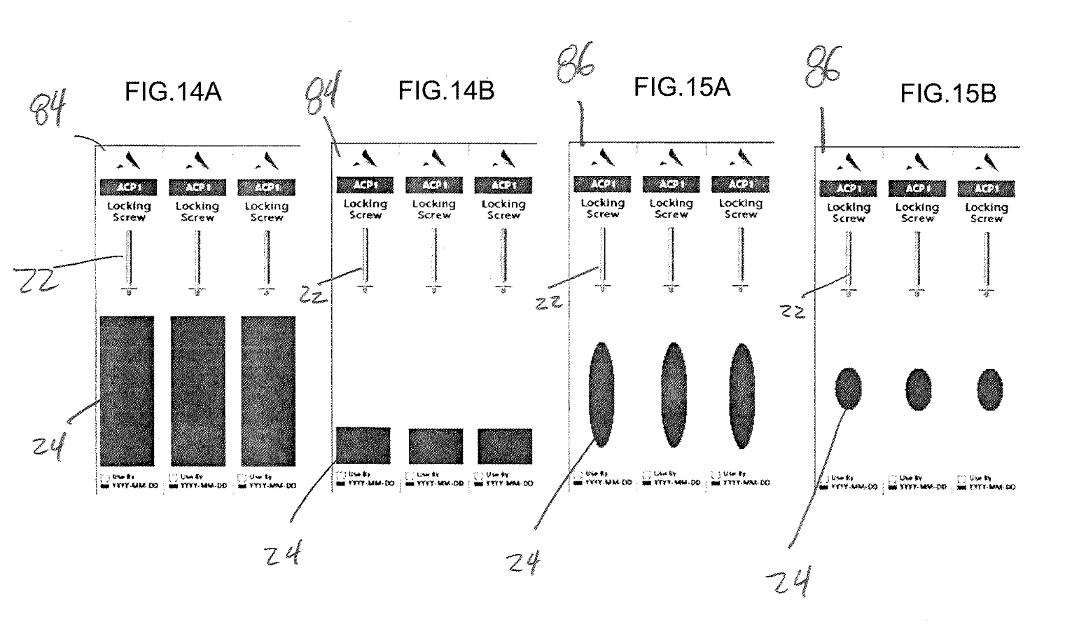

FIGS. 12A-12C illustrate a label 80 having a circle as the colored marker 24. FIGS. 13A-13C illustrate a label 82 having a triangle as the colored marker 24. FIGS. 14A-14B illustrate a label 84 having a solid bar as the colored marker 24. FIGS. 15A-15B illustrate a label 86 having an oval as the colored marker 24. Similarly to the labels described above, the size of the colored markers 24 of FIGS. 12A-15B corresponds to at least one property of the contents of the package (e.g. diameter, height, footprint, porosity, material composition). Labels having different shapes as the colored markers further visually distinguish the labels from each other and allow easy identification of package contents when the containers are positioned adjacent one another.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A label on a container with a medical device placed therein comprising:
a first indicator representative of the medical device in the container;
a second indicator representative of a first feature of the medical device, wherein the second indicator includes at least one marker of a first opacity and at least one marker of a second opacity such that the least or most opaque marker represents a dimension of the medical device, and
wherein a quantity of the markers represents a size of the medical device relative to other medical devices within a common product line.

2. The label of claim 1, wherein the first indicator is an image of the medical device.

3. The label of claim 1, wherein the at least one marker of the first opacity or the at least one marker of the second opacity is a colored marker.

4. The label of claim 3, wherein the number of colored markers are indicative of a feature of the medical device.

5. (canceled)

6. The label of claim 3, wherein the color of the colored marker indicates at least one of a diameter, height, and footprint of the medical device.

7. The label of claim 1, wherein the label comprises a plurality of faces, each face including the first indicator and the second indicator, each of the plurality of faces corresponding to a different face of the container.

8. The label of claim 1, wherein the label comprises an adhesive backing configured to be secured to the container.

9. The label of claim 1, further comprising a third indicator representative of a second feature of the medical device.

10. The label of claim 9, further comprising a fourth indicator indicative of a third feature of the medical device.

11. The label of claim 10, wherein the third indicator and the fourth indicator each signify at least one of a variable angle anchor, a self-drilling anchor, a fixed angle anchor, a self-tapping anchor, and a plate level.

12. The label of claim 1, further comprising a fifth indicator signifying a use-by date of the medical device.

13. A label on a container with a medical device placed therein comprising:
a first indicator representative of the medical device in the container; and
a second indicator representative of a first feature of the medical device;
wherein the label has an edge and the distance of the second indicator from the edge indicates a dimension and a comparative dimension of the first feature of the medical device, wherein the comparative dimension is based on a comparison between the medical device and other medical devices within a common product line.

14. The label of claim 13, wherein the first indicator is an image of the medical device.

15. The label of claim 13, wherein the second indicator includes a colored marker.

16. The label of claim 13, further comprising a third indicator representative of a second feature of the medical device.

17. The label of claim 16, further comprising a fourth indicator indicating a third feature of the medical device.

18. The label of claim 17, wherein the third indicator and the fourth indicator each signify at least one of a medical device dimension, a quantity, a variable angle anchor, a self-drilling anchor, a fixed angle anchor, a self-tapping anchor, and a plate level.

19. The label of claim 14, wherein the image portrays the quantity of the medical device in the container.

20. The label of claim 15, wherein the color of the colored marker indicates a diameter of the medical device.

21. A system for identification of container contents comprising:
a medical device placed in a container; and
a label placed or located on the container, the label comprising:
an indicator representative of a dimension of the medical device in the container relative to a smallest and a largest dimension of the medical device in a product line for the medical device;
wherein the label is distinguishable relative to a second label including a second indicator representative of a second medical device having a second dimension.
